(19) United States
(12) Patent Application Publication
(10) Pub. No.: US 2020/0032648 A1
(43) Pub. Date: Jan. 30, 2020

(54) SENSING SYSTEMS AND METHODS FOR DETECTING CHANGES IN DOWNHOLE HYDROCARBON AND GAS SPECIES

(71) Applicants: Weijia LU, Niskayuna,, NY (US); Yi LIAO, Niskayuna,, NY (US); General Electric Company, Schenectady, NY (US)

(72) Inventors: Weijia Lu, Shanghai (CN); Yi Liao, Oklahoma City, OK (US)

(73) Assignee: General Electric Company, Schenectady, NY (US)

(21) Appl. No.: 16/496,694

(22) PCT Filed: Mar. 23, 2017
(86) PCT No.: PCT/CN2017/077883

§ 371 Date: Sep. 23, 2019

(51) Int. Cl. E21B 49/08; G01N 33/00

(52) U.S. Cl. CPC E21B 49/081; E21B 47/06; G01N 33/0009

(57) ABSTRACT

A sensing system for a resource recovery system is provided. The sensing system includes at least one sensing sub-assembly and a sensing computing device. The sensing computing device is configured to receive, from the at least one sensing sub-assembly, at least one signal that includes at least one pulse having at least one pulse peak. The sensing computing device is also configured to identify the at least one pulse peak which has a magnitude and a signal-to-noise ratio, and retrieve the at least one pulse peak from the at least one signal using the magnitude and the signal-to-noise ratio of the at least one pulse peak. The sensing computing device is further configured to store the at least one pulse peak within a database that includes one or more pulse peaks, and generate a component report that identifies one or more changes of at least one component.

### BACKGROUND

The present disclosure relates generally to wellbore drilling and formation evaluation and, more specifically, to sensing systems for downhole hydrocarbon and gas species detection when forming a wellbore in a subterranean rock formation.

Hydraulic fracturing, commonly known as fracking, is a technique used to release petroleum, natural gas, and other hydrocarbon-based substances for extraction from underground reservoir rock formations, especially for unconventional reservoirs. The technique includes drilling a wellbore into the rock formations, and pumping a treatment fluid into the wellbore, which causes fractures to form in the rock formations and allows for the release of trapped substances produced from these subterranean natural reservoirs. At least some known unconventional subterranean wells are evenly fractured along the length of the wellbore. However, typically less than 50 percent of the fractures formed in the rock formations contribute to hydrocarbon extraction and production for the well. As such, hydrocarbon extraction from the well is limited, and significant cost and effort is expended for completing non-producing fractures in the wellbore.

### BRIEF DESCRIPTION

In one aspect, a sensing system for a resource recovery system is provided. The sensing system includes at least one sensing sub-assembly, and a sensing computing device. The sensing computing device is configured to receive, from the at least one sensing sub-assembly, at least one signal that includes at least one pulse having at least one pulse peak. The sensing computing device is also configured to identify the at least one pulse peak which has a magnitude and a signal-to-noise ratio, and retrieve the at least one pulse peak from the at least one signal using the magnitude and the signal-to-noise ratio of the at least one pulse peak. The sensing computing device is further configured to store the at least one pulse peak within a database that includes one or more pulse peaks, and generate a component report that identifies one or more changes of at least one component.

In a further aspect, a computer-implemented method for detecting changes of one or more components in a drilling fluid is provided. The method includes receiving, from at least one sensing sub-assembly, at least one signal that includes at least one pulse having at least one pulse peak. The method also includes identifying the at least one pulse peak which has a magnitude and a signal-to-noise ratio, and retrieving the at least one pulse peak from the at least one signal using the magnitude and the signal-to-noise ratio of the at least one pulse peak. The method further includes storing the at least one pulse peak within a database that includes one or more pulse peaks, and generating a component report that identifies one or more changes of at least one component.

In another aspect, a non-transitory computer readable medium that includes executable instructions for detecting changes of one or more components in a drilling fluid is provided. The computer executable instructions cause the sensing computing device to receive, from at least one sensing sub-assembly, at least one signal that includes at least one pulse having at least one pulse peak. The computer executable instructions also cause the sensing computing device to identify the at least one pulse peak which has a magnitude and a signal-to-noise ratio, and retrieve the at least one pulse peak from the at least one signal using the magnitude and the signal-to-noise ratio of the at least one pulse peak. The computer executable instructions further cause the sensing computing device to store the at least one pulse peak within a database that includes one or more pulse peaks, and generate a component report that identifies one or more changes of at least one component.

### DRAWINGS

These and other features, aspects, and advantages of the present disclosure will become better understood when the following detailed description is read with reference to the accompanying drawings in which like characters represent like parts throughout the drawings, wherein:

FIG. 1 is a block diagram of an exemplary computing device;

FIG. 2 is a block diagram of a portion of an exemplary sensing system that may include the computing device shown in FIG. 1;

FIG. 3 is a schematic illustration of an exemplary hydraulic fracturing system, including a drilling assembly, that may be used to form a wellbore;

FIG. 4 is a perspective view of an exemplary sensing sub-assembly that may be used in the drilling assembly shown in FIG. 3;

FIG. 5 is a perspective view of an exemplary sensing hub that may be used with the sensing sub-assembly shown in FIG. 4;

FIG. 6 is a schematic cross-sectional view of the sensing hub shown in FIG. 5, taken along Line 6-6;

FIG. 7 is exemplary method 700 of monitoring and detecting changes in downhole drilling fluid components using sensing system 200 (shown in FIG. 2); and

FIG. 8 is an exemplary method 800 of monitoring and detecting changes in downhole drilling fluid components using sensing system 200 (shown in FIG. 2).

Unless otherwise indicated, the drawings provided herein are meant to illustrate features of embodiments of the disclosure. These features are believed to be applicable in a wide variety of systems comprising one or more embodiments of the disclosure. As such, the drawings are not meant to include all conventional features known by those of ordinary skill in the art to be required for the practice of the embodiments disclosed herein.

### DETAILED DESCRIPTION

In the following specification and the claims, reference will be made to a number of terms, which shall be defined to have the following meanings.

The singular forms “a”, “an”, and “the” include plural references unless the context clearly dictates otherwise.

“Optional” or “optionally” means that the subsequently described event or circumstance may or may not occur, and that the description includes instances where the event occurs and instances where it does not.

Approximating language, as used herein throughout the specification and claims, may be applied to modify any quantitative representation that could permissibly vary without resulting in a change in the basic function to which it is related. Accordingly, a value modified by a term or terms, such as “about”, “approximately”, and “substantially”, are not to be limited to the precise value specified. In at least some instances, the approximating language may correspond to the precision of an instrument for measuring the value. Here and throughout the specification and claims, range limitations may be combined and/or interchanged. Such ranges are identified and include all the sub-ranges contained therein unless context or language indicates otherwise.

As used herein, the terms “processor” and “computer,” and related terms, e.g., “processing device,” “computing device,” and “controller” are not limited to just those integrated circuits referred to in the art as a computer, but broadly refers to a microcontroller, a microcomputer, a programmable logic controller (PLC), and application specific integrated circuit, and other programmable circuits, and these terms are used interchangeably herein. In the embodiments described herein, memory may include, but it not limited to, a computer-readable medium, such as a random access memory (RAM), and/or a computer-readable non-volatile medium, such as a flash memory. Alternatively, a floppy disk, a compact disc-read only memory (CD-ROM), a magneto-optical disk (MOD), and/or a digital versatile disc (DVD) may also be used. Also, in the embodiments described herein, additional input channels may be, but are not limited to, computer peripherals associated with an operator interface such as a mouse and a keyboard. Alternatively, other computer peripherals may also be used that may include, for example, but not be limited to, a scanner. Furthermore, in the exemplary embodiment, additional output channels may include, but not be limited to, an operator interface monitor.

Further, as used herein, the terms “software” and “firmware” are interchangeable, and include any computer program storage in memory for execution by personal computers, workstations, clients, and servers.

As used herein, the term “non-transitory computer-readable media” is intended to be representative of any tangible computer-based device implemented in any method of technology for short-term and long-term storage of information, such as, computer-readable instructions, data structures, program modules and sub-modules, or other data in any device. Therefore, the methods described herein may be encoded as executable instructions embodied in a tangible, non-transitory, computer-readable medium, including, without limitation, a storage device and/or a memory device. Such instructions, when executed by a processor, cause the processor to perform at least a portion of the methods described herein. Moreover, as used herein, the term “non-transitory computer-readable media” includes all tangible, computer-readable media, including, without limitation, non-transitory computer storage devices, including without limitation, volatile and non-volatile media, and removable and non-removable media such as firmware, physical and virtual storage, CD-ROMS, DVDs, and any other digital source such as a network or the Internet, as well as yet to be developed digital means, with the sole exception being transitory, propagating signal.

Furthermore, as used herein, the term “real-time” refers to at least one of the time of occurrence of the associated events, the time of measurement and collection of predetermined data, the time to process the data, and the time of a system response to the events and the environment. In the embodiments described herein, these activities and events occur substantially instantaneously.

Embodiments of the present disclosure relate to a sensing system for detecting changes of downhole drilling fluid components when forming a wellbore in a subterranean rock formation. The sensing system is implemented as a standalone evaluation system or installed as part of a wellbore drilling assembly. The sensing system determines characteristics of a first fluid discharged from the drilling assembly, and a second fluid that flows past the drilling assembly in the wellbore. More specifically, the sensing system includes a recessed cavity that receives a continuous stream of the second fluid, separate from the main flow of the second fluid. At least one sensor is positioned within the recessed cavity to facilitate protecting the sensor from the caustic and abrasive wellbore environment. The sensor is used to determine characteristics of the fluid, and the characteristics are analyzed to determine the changes of components in the fluid. As such, the analysis results are used to identify potentially promising fracture initiation zones within the wellbore such that efficient and cost effective completion planning can be implemented.

For example, detection of changes of downhole drilling fluid components while drilling facilitates identifying zones of high permeability, such as open natural fractures, clusters of closed but unsealed natural fractures, larger pores, and other formation features where hydrocarbons are stored. The analysis results can be used to identify the most promising fracture initiation points or zones, and the information can be used for completion planning, especially for unconventional reservoirs. In addition, the analysis results can be used to identify poor zones (no gas show), which facilitates reducing the time and effort of perforating and stimulating the poor zones. Another potential application is for geosteering assistance, wherein the real time gas show/species information is used to adjust the borehole position (e.g., inclination and azimuth angles) while drilling, such that a well having increased production can be formed. Finally, the sensing system can also provide kick detection to facilitate providing real-time alerts of gas flow potential for safety and environmental considerations, thereby reducing the risk of system failure.

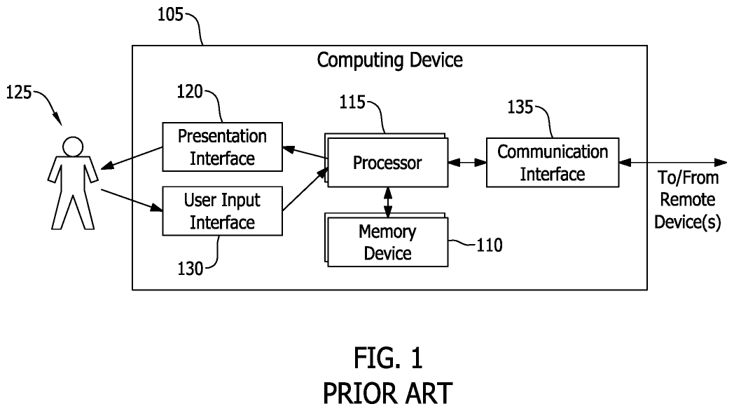

FIG. 1 is a block diagram of an exemplary computing device 105 that may be used to perform monitoring and/or detection of a sensing system (not shown in FIG. 1) and, more specifically, facilitate identifying changes of downhole drilling fluid components (not shown in FIG. 1) within a wellbore. Also, in the exemplary embodiment, computing device 105 monitors and/or controls any piece of equipment, any system, and any process associated with the sensing system, e.g., without limitation, sensing sub-assemblies, sensors, acoustic probes, ultrasonic probes, and various sensing devices (neither shown in FIG. 1) for the sensing system. Computing device 105 includes a memory device 110 and a processor 115 operatively coupled to memory device 110 for executing instructions. In some embodiments, executable instructions are stored in memory device 110. Computing device 105 is configurable to perform one or more operations described herein by programming processor 115. For example, processor 115 may be programmed by encoding an operation as one or more executable instructions and providing the executable instructions in memory device 110. In the exemplary embodiment, memory device 110 is one or more devices that enable storage and retrieval of information such as executable instructions and/or other data. Memory device 110 may include one or more computer readable media.

Memory device 110 may be configured to store operational measurements including, without limitation, real-time and historical signal data of the sensing system and/or any other type data. Also, memory device 110 includes, without limitation, sufficient data, algorithms, and commands to facilitate monitoring and detecting changes of downhole drilling fluids.

In some embodiments, computing device 105 includes a presentation interface 120 coupled to processor 115. Presentation interface 120 presents information, such as a user interface and/or an alarm, to a user 125. In some embodiments, presentation interface 120 includes one or more display devices. In some embodiments, presentation interface 120 presents an alarm associated with the associated electric power distribution system being monitored and controlled, such as by using a human machine interface (HMI) (not shown in FIG. 1). Also, in some embodiments, computing device 105 includes a user input interface 130. In the exemplary embodiment, user input interface 130 is coupled to processor 115 and receives input from user 125.

A communication interface 135 is coupled to processor 115 and is configured to be coupled in communication with one or more other devices, such as a sensor or another computing device 105, and to perform input and output operations with respect to such devices while performing as an input channel. Communication interface 135 may receive data from and/or transmit data to one or more remote devices. For example, a communication interface 135 of one computing device 105 may transmit an alarm to the communication interface 135 of another computing device 105.

In the exemplary embodiment, detecting and monitoring of a sensing system is performed with local control devices, i.e., a localized computing device 105. Alternatively, detecting and monitoring of such sensing systems may be performed as a portion of a larger, more comprehensive system.

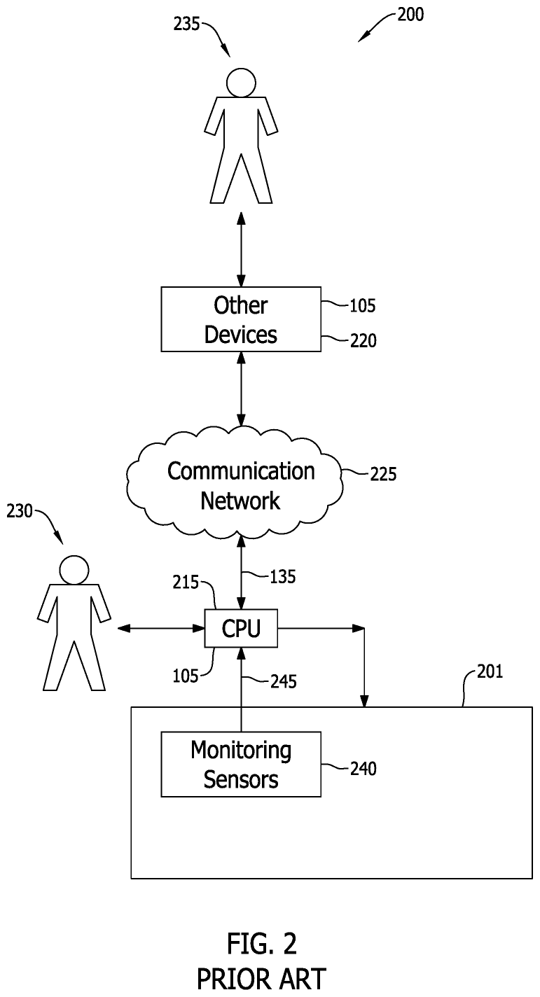

FIG. 2 is a block diagram of a portion of a sensing system 200 that may be used to monitor and detect at least a portion of a sensing sub-assembly system 201. Sensing system 200 includes at least one central processing unit (CPU) 215 configured to execute monitoring and detecting algorithms and monitoring and detecting logic. CPU 215 may be coupled to other devices 220 via a communication network 225, where, in some embodiments, communication network 225 includes the Internet. In some embodiments, CPU 215 is a computing device 105. In other embodiments, CPU 215 is a controller.

CPU 215 interacts with a first operator 230, e.g., without limitation, via user input interface 130 and/or presentation interface 120 (both shown in FIG. 1). In one embodiment, CPU 215 presents information about sensing sub-assembly 201, such as alarms, to operator 230. Other devices 220 interact with a second operator 235, e.g., without limitation, via user input interface 130 and/or presentation interface 120. For example, other devices 220 present alarms and/or other operational information to second operator 235. As used herein, the term “operator” includes any person in any capacity associated with operating and maintaining sensing sub-assembly 201.

In some embodiments, other devices 220 include one or more storage devices that are any computer-operated hardware suitable for storing and/or retrieving data, for example, and without limitation, multiple storage units such as hard disks or solid state disks in a redundant array of inexpensive disks (RAID) configuration, a storage area network (SAN), and/or a network attached storage (NAS) system.

Sensing sub-assembly 201 includes one or more monitoring sensors 240 coupled to CPU 215 through at least one input channel 245. Monitoring sensors 240 collect operational measurements including, without limitation, viscosity changes, compressibility changes, and/or type of components in downhole drilling fluids in contact with mechanical devices of sensing sub-assembly 201. Monitoring sensors 240 repeatedly, e.g., periodically, continuously, and/or upon request, transmit operational measurement readings at the time of measurement. CPU 215 receives and processes the operational measurement readings. In one embodiment, such data may be transmitted across network 225 and may be accessed by any device capable of accessing network 225 including, without limitation, desktop computers, laptop computers, and personal digital assistants (PDAs) (neither shown).

In some embodiments, some components described for FIG. 2 may be used with the stand-alone computing device 105 (as shown in FIG. 1), e.g., without limitation, monitoring sensors 240. In such embodiments, computing device 105 includes, without limitation, sufficient data, algorithms, and commands to independently facilitate detecting and monitoring of sensing sub-assembly 201 as described herein, thereby embodying a significant portion of sensing system 200 substantially within stand-alone computing device 105.

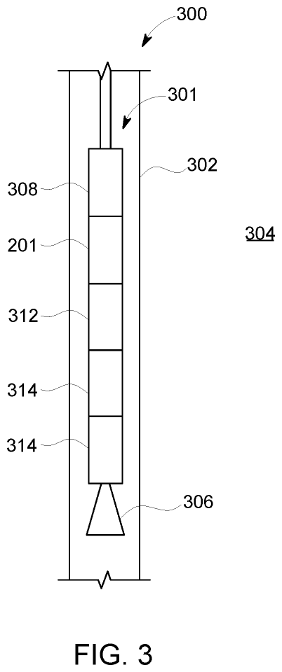

FIG. 3 is a schematic illustration of an exemplary hydraulic fracturing system 300. Hydraulic fracturing system 300 includes a drilling assembly 301 that may be used to form a wellbore 302 in a subterranean rock formation 304. In the exemplary embodiment, drilling assembly 301 includes a plurality of sub-assemblies and a drill bit 306. More specifically, the plurality of sub-assemblies include a measurement-while-drilling or logging-while-drilling sub-assembly 308, a sensing sub-assembly 201, a mud motor 312, and bent housing or rotary steerable system sub-assemblies 314 coupled together in series. Drilling assembly 301 includes any arrangement of sub-assemblies that enables drilling assembly 301 to function as described herein.

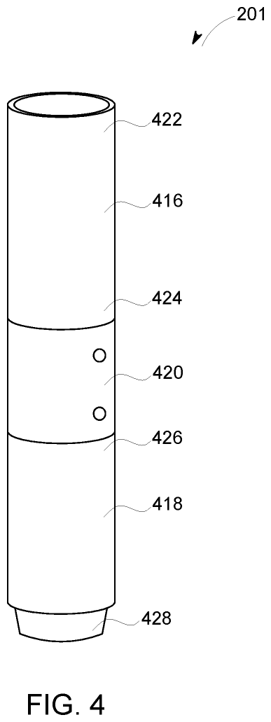

FIG. 4 is a perspective view of sensing sub-assembly 201 that may be used in drilling assembly 301 (shown in FIG. 3). In the exemplary embodiment, sensing sub-assembly 201 includes a first outer casing 416, a second outer casing 418, and a sensing hub 420 coupled therebetween. First outer casing 416 includes a first end 422 and a second end 424, and second outer casing 418 includes a first end 426 and a second end 428. First end 422, second end 424, first end 426, and second end 428 each include a threaded connection for coupling sensing sub-assembly 201 to one or more of the plurality of sub-assemblies of drilling assembly 301, and for coupling first outer casing 416 and second outer casing 418 to sensing hub 420.

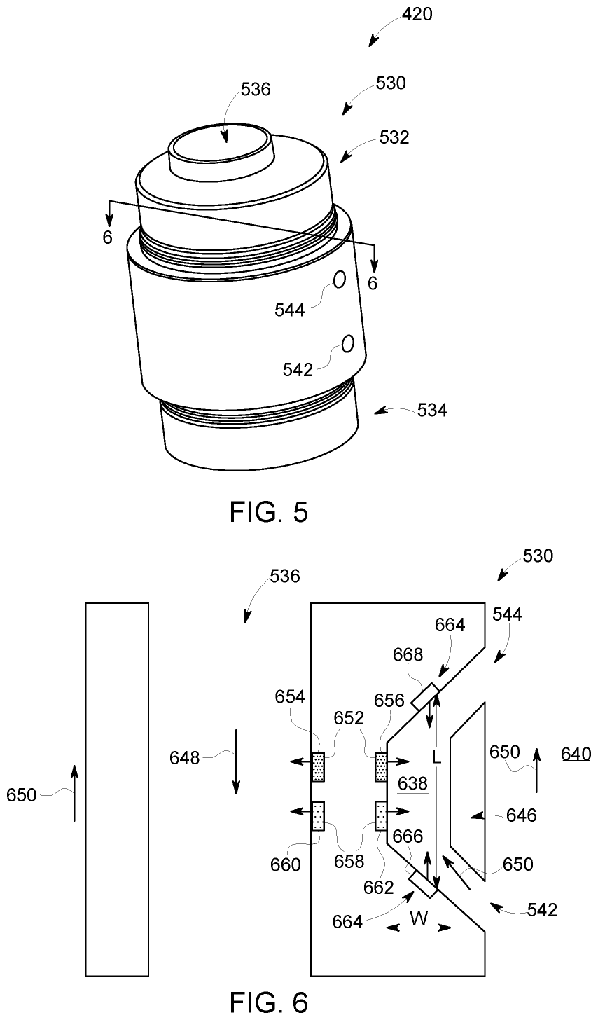

FIG. 5 is a perspective view of sensing hub 420 (shown in FIG. 4) that may be used with sensing sub-assembly 201 (shown in FIG. 4), and FIG. 6 is a schematic cross-sectional view of sensing hub 420, taken along Line 6-6 (shown in FIG. 5). In the exemplary embodiment, sensing hub 420 includes a cylindrical body 530 including a first end 532 and a second end 534. First end 532 and second end 534 each include a threaded connection for coupling to first outer casing 416 and second outer casing 418 (both shown in FIG. 4), as described above. In addition, cylindrical body 530 includes an internal flow channel 536 extending therethrough that channels high pressure fluid during operation of drilling assembly 301, as will be described in more detail below.

Cylindrical body 530 further includes a recessed cavity 638 defined therein. Recessed cavity 638 is either at least partially obstructed from or fully exposed to an ambient environment 640 exterior of cylindrical body 530, as will be explained in more detail below. As shown in FIG. 6, recessed cavity 638 is at least partially obstructed from ambient environment 640. More specifically, cylindrical body 530 further includes a flow inlet 542 and a flow outlet 544 defined therein, such that an outer portion 646 of cylindrical body 530 is positioned between recessed cavity 638 and ambient environment 640. In addition, recessed cavity 638 extends longitudinally parallel with internal flow channel 536 (shown in FIG. 5). Alternatively, recessed cavity 638 extends helically within cylindrical body 530 to increase the flexibility for sensor arrangement within recessed cavity 638.

During operation of drilling assembly 301 (shown in FIG. 3), a first fluid 648 is channeled through internal flow channel 536, and is discharged from drilling assembly 301, and a second fluid 650 backflows within wellbore 302 (shown in FIG. 3) past drilling assembly 301. First fluid 648 flows at a greater pressure than second fluid 650, and second fluid 650 includes a portion of first fluid 648 and constituents of subterranean rock formation 304. Referring to FIG. 6, in one embodiment, recessed cavity 638 receives a continuous stream of second fluid 650. More specifically, flow inlet 542 channels the continuous stream of second fluid 650 into recessed cavity 638, and flow outlet 544 discharges the continuous stream of second fluid 650 from recessed cavity 638. As such, second fluid 650 is channeled through recessed cavity 638 for further analysis either continuously or at predetermined intervals, as will be explained in more detail below.

In the exemplary embodiment, sensing sub-assembly 201 (shown in FIG. 4) includes at least one sensor coupled to cylindrical body 530. As will be described in further detail below, the at least one sensor determines characteristics of first fluid 648 and second fluid 650, and the data obtained from the at least one sensor is transmitted to computing device 105 (shown in FIG. 2) to determine the drilling fluid content of second fluid 650. Exemplary characteristics determined by the at least one sensor include, but are not limited to, density, viscosity, sound speed, sound attenuation, and attenuation coefficient. Exemplary sensors include, but are not limited to, an ultrasound sensor, and an acoustic sensor, such as an acoustic transducer. Alternatively, any sensors for determining characteristics of first fluid 648 and second fluid 650 may be utilized that enables sensing sub-assembly 201 to function as described herein.

In one embodiment, referring to FIG. 6, the at least one sensor includes a first pair 652 of sensors, including a first sensor 654 and a second sensor 656. First sensor 654 is positioned for determining characteristics of first fluid 648 within internal flow channel 536, and second sensor 656 is positioned for determining characteristics of second fluid 650 within recessed cavity 638. First pair 652 of sensors operate at the same frequency, such that the data obtained from first sensor 654 and second sensor 656 are comparable relative to each other for determining the hydrocarbon content in second fluid 650, as will be explained in more detail below. In one embodiment, first sensor 654 and second sensor 656 are wide band transducers that operate at a frequency defined within a range between and including about 100 kilohertz (kHz) and about 2.5 megahertz (MHz).

Alternatively, the at least one sensor includes first pair 652 of sensors and a second pair 658 of sensors, including a third sensor 660 and a fourth sensor 662. Similar to first pair 652 of sensors, third sensor 660 is positioned for determining characteristics of first fluid 648 within internal flow channel 536, and fourth sensor 662 is positioned for determining characteristics of second fluid 650 within recessed cavity 638. In addition, first pair 652 of sensors operates at the same first frequency, and second pair 658 of sensors operates at the same second frequency. In the exemplary embodiment, the operating frequencies of first pair 652 and second pair 658 are defined within a sub-range of the operating frequency of the wide band transducer described above (i.e., sub-ranges spanning a portion of the range defined between and including about 100 kHz and about 2.5 MHz that collectively span a wide frequency range. As such, the data obtained from third sensor 660 and fourth sensor 662 are comparable relative to each other for determining the hydrocarbon content in second fluid 650.

In some embodiments, the at least one sensor further includes a third pair 664 of sensors, including a fifth sensor 666 and a sixth sensor 668. Fifth sensor 666 and sixth sensor 668 are positioned for determining characteristics of second fluid 650 within recessed cavity 638. More specifically, one of fifth sensor 666 and sixth sensor 668 is an emitter, and the other sensor is a receiver. In addition, recessed cavity 638 has a length L and a width W shorter than length L. Fifth sensor 666 and sixth sensor 668 are longitudinally spaced from each other within recessed cavity 638 relative to length L. Longitudinally spacing fifth sensor 666 and sixth sensor 668 from each other facilitates increasing the distance therebetween, such that the distance is not limited by the diameter of cylindrical body 530. Moreover, in one embodiment, fifth sensor 666 and sixth sensor 668 are low frequency transducers that operate at a frequency defined within a range between and including about 10 kHz and about 20 kHz. When compared to higher frequency transducers, the sensor readings obtained from third pair 664 of sensors are less likely to be scattered by gas bubbles contained in second fluid 650, for example. As such, operating third pair 664 of sensors at a low frequency range facilitates increasing the amount of useful data obtained for later analysis and evaluation, by computing device 105 (shown in FIG. 1), to determine the content of second fluid 650.

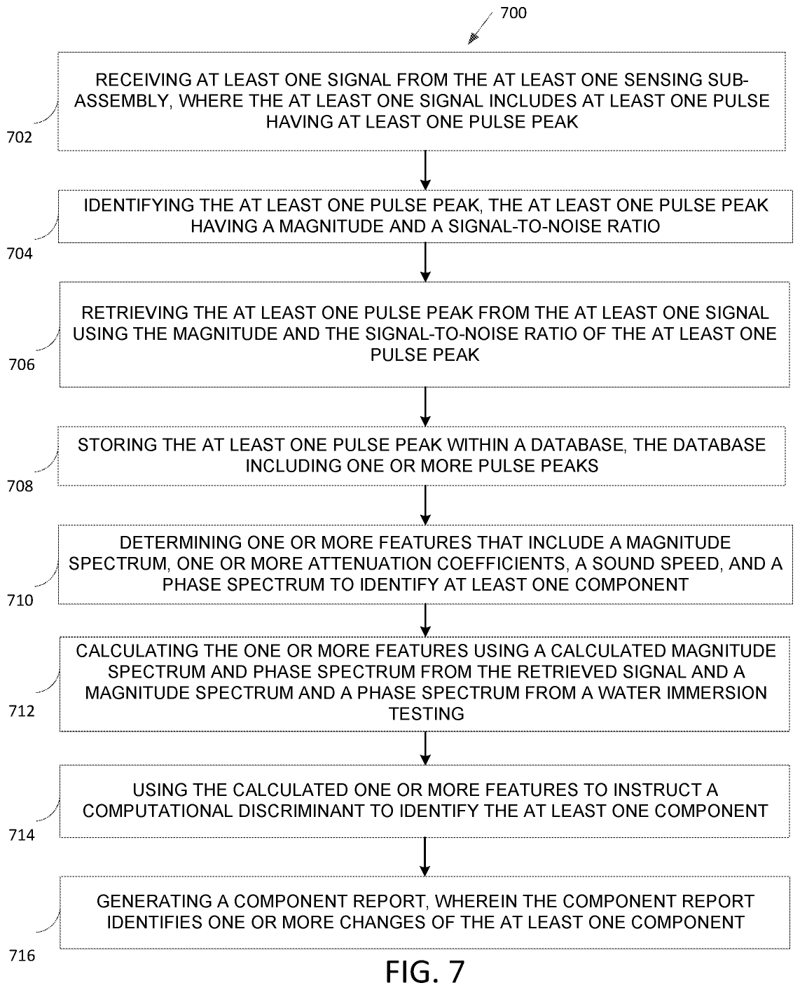

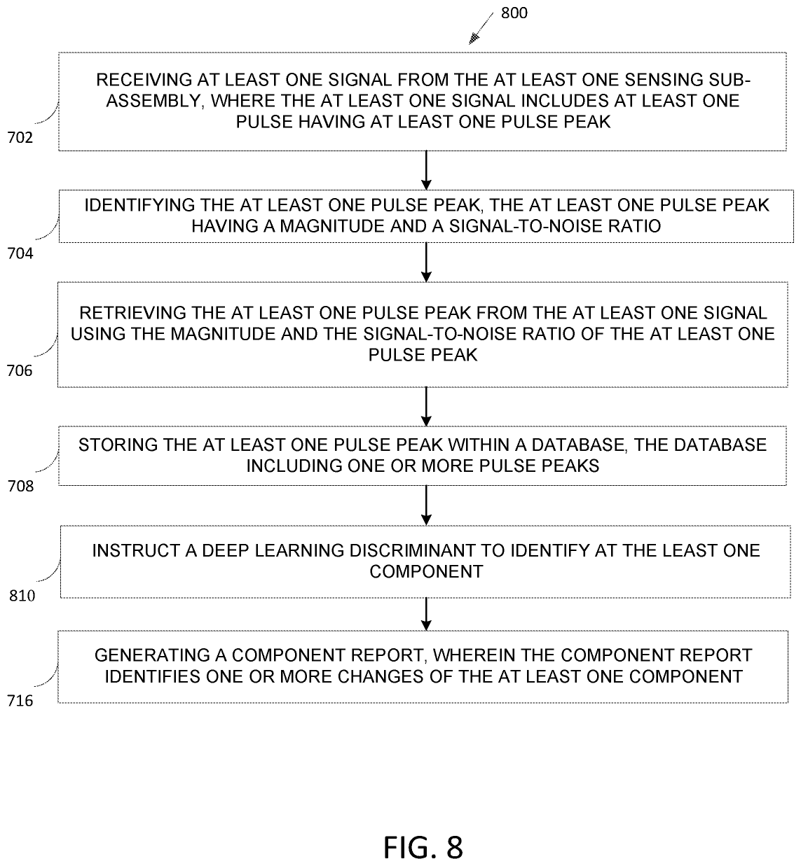

FIG. 7 and FIG. 8 are exemplary methods 700 and 800 of monitoring and detecting changes in downhole drilling fluid components using sensing system 200 (shown in FIG. 2). Sensing system 200 includes sensing sub-assembly 201 (shown in FIGS. 3-6) coupled to computing device 105 (shown in FIGS. 1 and 2). In method 700, computing device 105 uses a computational discriminant to perform at least a portion of method 700, whereas in method 800, computing device 105 uses a deep learning discriminant to perform at least portion of method 800. Referring to FIGS. 1-7, methods 700 and 800 include receiving 702, from sensing sub-assembly device 201, one or more signals that include at least one pulse having at least one pulse peak. Sensing sub-assembly device 201 includes a plurality of sensors 652, 654, 656, 658, 660, 662, 666, 668, configured to collect the one or more signals. In alternative embodiments, the one or more signals include a pulse train having a plurality of pulses. Methods 700 and 800 also include identifying 704 the at least one pulse peak which has a magnitude and a signal-to-noise ratio. In some embodiments, methods 700 and 800 include identifying a first pulse peak when the one or more signals include a pulse train. In other embodiments, method 700 and 800 include identifying the highest magnitude of the plurality of magnitudes in the at least one pulse.

Methods 700 and 800 further include retrieving 706 the at least one pulse peak from the one or more signals using the magnitude and the signal-to-noise ratio. In alternative embodiments, methods 700 and 800 include removing at least one pulse peak that is not retrieved from the one or more signals included in the pulse train. Methods 700 and 800 also include storing 708 the at least one pulse peak within a database, the database including one or more pulse peaks.

Referring to FIG. 7, method 700 includes determining 710 one or more features, where the one or more features include a magnitude spectrum, one or more attenuation coefficients, a sound speed, and a phase spectrum to identify at least one component. In the exemplary embodiment, method 700 determines the one or more features from the received at least one signal.

For example, in method 700 determining 710 may include calculating a magnitude spectrum and a phase spectrum from the at least one pulse peak, and a sound speed with respect to a time the at least one pulse peak was received. Method 700 also may include calculating 712 the one or more features using the calculated magnitude spectrum and phase spectrum from the retrieved signal and a magnitude spectrum and a phase spectrum from a water immersion testing. Calculating 712 the one or more features may at least include:
a magnitude attenuation (α) on each frequency and from which one or more attenuation coefficients may be estimated;
a mean frequency, namely the expectation of the frequency on the distribution of α;
relative frequency to the peaks on the α;
a bandwidth of the α, namely the 2nd central moment of the frequency on the distribution of the α;
a phase spectrum (φ) change on each frequency, from which a polynomial function may be determined, and use coefficients of the polynomial function to denote a viscosity change; and
descriptive statistics on α and φ: mean, standard, skewness, or the like.

Method 700 may use any suitable signal processing method to calculate features mentioned above, such as, for example, a subband decomposing and reconstructing signal by wavelet.

Method 700 also includes using 714 the calculated one or more feature to instruct a computational discriminant model to identify at least one component. The output of the computational discriminant model includes at least one predetermined component type. The method for designing the computational discriminant model may include, but is not limited to, randomly partitioning the storage signal in a database, automatically tuning the super parameters on at least one computational discriminant model, quantifying metric and automatically voting the computational discriminant model. Method 700 may also include overwhelming, overfitting, maintaining, and/or managing the computational discriminant model version in the database, as well as online learning and updating the computational discriminant model once new retrieved signals are received reinforcement learning. The computational discriminant model may include, but is not limited to, supervised machine learning models, such as LDA, logistic regression, SVM, decision tree, random forest, or the like.

Referring to FIG. 8, in comparison to method 700, method 800 includes instructing 810 a deep learning model to identify at least one component. The deep learning model identifies the at least one component by instructing directly on the signal instead of using the calculated features, mentioned in method 700, when a certain scale of sample in database is met. The instructions for designing the deep learning model may include, but are not limited to, instructions for designing the discriminant model in previous entry. Further, the deep learning model may be, but is not limited to, a convolutional neural network (CNN or ConvNet) to learn features, and an optional recurrent neural network (RNN) to catch up feature transformation in a temporal range, or the like.

Methods 700 and 800 further include generating 716 a component report that identifies one or more changes of at the least one component. The component report may also include the one or more of features of the at least one signal.

The systems and assemblies described herein facilitate providing at least semi-continuous hydrocarbon and gas species detection feedback when drilling unconventional subterranean wells. More specifically, the drilling assembly facilitates analyzing fluid used in the drilling process in a fast and efficient manner. The data obtained from the analysis of the fluid samples can then be used to determine zones within a wellbore that have either a low likelihood or a high likelihood of having high hydrocarbon content. As such, the zones having high hydrocarbon content are identified, and fracture completion planning resulting in improved well production is determined.

An exemplary technical effect of the systems and assemblies described herein includes at least one of: (a) providing real-time and continuous downhole drilling fluid components state detection feedback when forming a well in a subterranean rock formation; (b) identifying potentially promising fracture initiation zones within a wellbore; (c) improving hydrocarbon production for wells; (d) providing geosteering assistance for the drilling assembly; and (e) providing kick detection for real-time gas flow potential safety alerts.

Exemplary embodiments of a drilling assembly and related components are described above in detail. The drilling assembly is not limited to the specific embodiments described herein, but rather, components of systems and/or steps of the methods may be utilized independently and separately from other components and/or steps described herein. For example, the configuration of components described herein may also be used in combination with other processes, and is not limited to practice with only drilling and sensing assemblies and related methods as described herein. Rather, the exemplary embodiment can be implemented and utilized in connection with many applications where analyzing one or more fluids is desired.

Although specific features of various embodiments of the present disclosure may be shown in some drawings and not in others, this is for convenience only. In accordance with the principles of embodiments of the present disclosure, any feature of a drawing may be referenced and/or claimed in combination with any feature of any other drawing.

Some embodiments involve the use of one or more electronic or computing devices. Such devices typically include a processor, processing device, or controller, such as a general purpose central processing unit (CPU), a graphics processing unit (GPU), a microcontroller, a reduced instruction set computer (RISC) processor, an application specific integrated circuit (ASIC), a programmable logic circuit (PLC), a field programmable gate array (FPGA), a digital signal processing (DSP) device, and/or any other circuit or processing device capable of executing the functions described herein. The methods described herein may be encoded as executable instructions embodied in a computer readable medium, including, without limitation, a storage device and/or a memory device. Such instructions, when executed by a processing device, cause the processing device to perform at least a portion of the methods described herein. The above examples are exemplary only, and thus are not intended to limit in any way the definition and/or meaning of the term processor and processing device.

This written description uses examples to disclose the embodiments, including the best mode, and also to enable any person skilled in the art to practice the embodiments, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A sensing system for a resource recovery system, said sensing system comprising:
at least one sensing sub-assembly including at least one pair of probes; and
a sensing computing device comprising a processing device and a memory coupled to said processing device, said sensing computing device in communication with said at least one sensing sub-assembly, said sensing computing device configured to:
receive at least one signal from said at least one sensing sub-assembly, wherein the at least one signal includes at least one pulse having at least one pulse peak;
identify the at least one pulse peak, the at least one pulse peak having a magnitude and a signal-to-noise ratio;
retrieve the at least one pulse peak from the at least one signal using the magnitude and the signal-to-noise ratio of the at least one pulse peak;
store the at least one pulse peak within a database, the database including one or more pulse peaks; and
generate a component report, wherein the component report identifies one or more changes of the at least one component.

2. The sensing system in accordance with claim 1, wherein said sensing computing device is further configured to:
determine that the signal includes a pulse train;
identify a pulse peak having a first position in the pulse train; and
retrieve the pulse peak having the first position in the pulse train.

3. The sensing system in accordance with claim 1, wherein said sensing computing device is further configured to:
compare a plurality of magnitudes in the at least one pulse;
identify, in the at least one pulse, a pulse peak having the highest magnitude of the plurality of magnitudes in the at least one pulse; and
retrieve the pulse peak.

4. The sensing system in accordance with claim 1, wherein said sensing computing is further configured to instruct a deep learning discriminant to identify the at least one component.

5. The sensing system in accordance with claim 1, wherein said sensing computing is further configured to determine one or more features, wherein the one or more features include a magnitude spectrum, one or more attenuation coefficients, a sound speed, and a phase spectrum to identify the at least one component.

6. The sensing system in accordance with claim 1, wherein said sensing computing is further configured to generate the component report, wherein the component report includes the one or more of features of the at least one signal.

7. The sensing system in accordance with claim 1, wherein said at least one sensing sub-assembly further comprises:
an internal flow conduit defined therein and extending therethrough, said internal flow conduit configured to channel a first fluid therethrough;
a recessed cavity defined therein, said recessed cavity coupled in flow communication with an ambient environment exterior of said at least one sensing sub-assembly, wherein a second fluid flows within the ambient environment, said recessed cavity configured to receive a continuous stream of the second fluid; and
at least one sensor configured to determine characteristics of the second fluid in the continuous stream that flows through said recessed cavity.

8. A computer-implemented method for detecting changes of one or more components in a drilling fluid, said method implemented using a sensing computing device, said method comprising:
receiving at least one signal from at least one sensing sub-assembly, wherein the at least one signal includes at least one pulse having at least one pulse peak;
identifying the at least one pulse peak, the at least one pulse peak having a magnitude and a signal-to-noise ratio;
retrieving the at least one pulse peak from the at least one signal using the magnitude and the signal-to-noise ratio of the at least one pulse peak;
storing the at least one pulse peak within a database, the database including one or more pulse peaks; and
generating a component report, wherein the component report identifies one or more changes of the at least one component.

9. The method in accordance with claim 8, wherein identifying the at least one pulse peak comprises:
determining that the signal includes a pulse train;
identifying a pulse peak having a first position in the pulse train; and
retrieving the pulse peak having the first position in the pulse train.

10. The method in accordance with claim 8, wherein identifying the at least one pulse peak comprises:
comparing a plurality of magnitudes in the at least one pulse;
identifying, in the at least one pulse, a pulse peak having the highest magnitude of the plurality of magnitudes in the at least one pulse; and
retrieving the pulse peak.

11. The method in accordance with claim 8 further comprising instructing a deep learning discriminant to identify the at least one component

12. The method in accordance with claim 8 further comprising determining one or more features, wherein the one or more features include a magnitude spectrum, one or more attenuation coefficients, a sound speed, and a phase spectrum to identify the at least one component.

13. The method in accordance with claim 12, wherein determining the one or more features includes calculating a magnitude spectrum and a phase spectrum from the at least one pulse peak, and a sound speed with respect to a time the at least one pulse peak was received.

14. The method in accordance with claim 13 further comprising calculating the one or more features using the calculated magnitude spectrum and phase spectrum from the retrieved signal and a magnitude spectrum and a phase spectrum from a water immersion testing.

15. The method in accordance with claim 8 further comprising generating the component report, wherein the component report includes the one or more of features of the at least one signal.

16. A non-transitory computer readable medium that includes executable instructions for detecting changes of one or more components in a drilling fluid, wherein when executed by a sensing computing device comprising at least one processing device, the computer executable instructions cause the sensing computing device to:
receive at least one signal from at least one sensing sub-assembly, wherein the at least one signal includes at least one pulse having at least one pulse peak;
identify the at least one pulse peak, the at least one pulse peak having a magnitude and a signal-to-noise ratio;
retrieve the at least one pulse peak from the at least one signal using the magnitude and the signal-to-noise ratio of the at least one pulse peak;
store the at least one pulse peak within a database, the database including one or more pulse peaks; and
generate a component report, wherein the component report identifies one or more changes of at least one component.

17. The computer readable medium in accordance with claim 16, wherein said computer executable instructions cause the sensing computing device to:
determine that the signal includes a pulse train;
identify a pulse peak having a first position in the pulse train; and
retrieve the pulse peak having the first position in the pulse train.

18. The computer readable medium in accordance with claim 16, wherein said computer executable instructions cause the sensing computing device to:
compare a plurality of magnitudes in the at least one pulse;
identify, in the at least one pulse, a pulse peak having the highest magnitude of the plurality of magnitudes in the at least one pulse; and
retrieve the pulse peak.

19. The computer readable medium in accordance with claim 16, wherein said computer executable instructions cause the sensing computing device to instruct a deep learning discriminant to identify the at least one component.

20. The computer readable medium in accordance with claim 16, wherein said computer executable instructions cause the sensing computing device to determine one or more features, wherein the one or more features include a magnitude spectrum, one or more attenuation coefficients, a sound speed, and a phase spectrum to identify the at least one component.
